(19) United States
(12) Patent Application Publication
(10) Pub. No.: US 2004/0228915 A1
(43) Pub. Date: Nov. 18, 2004

(54) Oral extended release compressed tablets of multiparticulates

(76) Inventors: Robert M. Noack, Ann Arbor, MI (US); John M. Heimlich, Portage, MI (US); Ernest J. Lee, Kalamazoo, MI (US)

Correspondence Address: Karen B. King, Warner-Lambert Company LLC, 2800 Plymouth Road, Ann Arbor, MI, 48105, US

(21) Appl. No.: 10/816,699

(22) Filed: Apr. 2, 2004

Related U.S. Application Data
(60) Provisional application No. 60/460,851, filed on Apr. 4, 2003.

(51) Int. Cl. A61K 31/704; A61K 9/26; A61K 9/24

(52) U.S. Cl. 424/471; 514/35

(57) ABSTRACT

The present invention is directed to an extended release multiparticulate formulation of a therapeutic agent, wherein coated core multiparticulate particles of the therapeutic agent are overcoated with a binder-dispersing agent, such as povidone or cross-povidone. The invention is also directed to compressed tablets of the extended release multiparticulate formulation of the invention, and to a method of oral administration of compressed tablets of clindamycin to a subject to treat or prevent a gram-positive bacterial infection therein. The binder-dispersing agent in the formulations of the present invention ensure that compressed tablets formed therefrom will remain intact through oral administration, and dissolve shortly thereafter, enabling the multiparticulates to release the therapeutic agent contained therein over an extended period of time.

### CROSS-REFERENCE TO RELATED APPLICATIONS

This application claims the benefit of U.S. Provisional Application Serial No. 60/460,851, filed Apr. 4, 2003.

### FIELD OF THE INVENTION

The present invention relates to oral extended release compressed tablets, and to methods of making the same. The present invention further relates to compressed tablets wherein the active agent is present in the form of multiparticulates coated with at least one polymer that extends the rate of release of the active agent from the multiparticulates.

### BACKGROUND

Multiparticulates have been used to extend the rate of delivery of therapeutic agents in a variety of different formulations, including suspensions, capsules, and compressed tablets. Of these three means of administration, compressed tablets have the greatest load capacity, something essential for oral delivery of therapeutic agents where a pharmaceutically effective dose is relatively large.

Various means of controlling the rate of delivery of therapeutic agents from multiparticulates have been developed. U.S. Pat. No. 6,248,363 (Patel et al.) discloses solid carrier compositions in the form of beads, granules, or particles coated with an enteric coating (U.S. Pat No. 6,248,363) or other hydrophilic surfactant. The solid carrier compositions comprise a substrate that can be an additive, an active ingredient, or a mixture thereof. (claim 20) The coating of the solid carrier of the '363 patent optionally includes a therapeutic agent. Several suitable coating materials are disclosed, including enteric coatings. When the coating includes a therapeutic agent, the patent indicates that quick release of the therapeutic agent is facilitated. Incorporation of at least some of the therapeutic agent into the solid carrier composition and coating with a release controlling polymer facilitates extended release of the therapeutic agent. The patent indicates that the coated carriers disclosed therein can be formulated into compressed tablets.

U.S. Patent Application Publication No. U.S. 2003/0026839 A1 discloses a different type of multiparticulates, produced by melt-extrusion. Such multiparticulates are produced by heating a therapeutic agent, a retardant, and an optional binder together and extruding the resulting mixture to produce sustained-release multiparticulate formulations. The resulting multiparticulates can allegedly be compressed into tablets. The retardant is preferably a hydrophobic polymer or alkylcellulose or water soluble hydroxyalkylcellulose with the capacity to retard release of the therapeutic agent from the multiparticulates. Ethylcellulose and Eudragit RS30D are cited as examples of retardants suitable for use in the melt-extruded multiparticulates. The application goes on to state that the melt-extruded multiparticulates may also be overcoated with an aqueous dispersion of the hydrophobic polymer, preferably, with an aqueous dispersion that also includes a plasticizer. Preformulated aqueous dispersions, such as Surelease® or Eudragit coating solutions may also be used. The resulting melt-extruded coated multiparticulates can be compressed and shaped into tablets.

Release modifying matrices in the core of multiparticulates have also been used to control the rate of release of a therapeutic agent therefrom. Examples of such matrices can be found in U.S. Pat. No. 6,517,866 (Am Ende et al.), which illustrates dosage forms of sertraline, including non-eroding matrices, hydrophobic eroding matrices, and coated matrix systems. The matrix systems of the '866 patent include those in which “sertraline is dissolved, embedded, or dispersed in a matrix of another material that serves to retard the release of sertraline into an aqueous environment (i.e., the lumenal fluid of the GI tract).” (col. 15, lines 33-36). When the sertraline dosage forms are coated, they are preferably in the form of a membrane-moderated reservoir system, such as a membrane-coated diffusion-based multiparticulate, tablet, or capsule. Rate of drug delivery is controlled by factors such as “the permeability and thickness of the coating, the osmotic pressure of the sertraline-containing layer, the water activity of the hydrogel layer, and the surface area of the device.” (Col. 26, lines 18-22).

U.S. Pat. No. 6,228,398 (Devane et al.) discloses multiple-pulsed release formulations that employ multiparticulates with at least two different polymer coatings or with at least two different thicknesses of polymer coatings. The formulations disclosed therein each have an immediate release component and at least one modified release component. A variety of different polymer coatings were disclosed, including Eudragit RS or RL. Polyvinylpyrrolidine (“PVP”) and hydroxypropylmethyl cellulose (“HPMC”) were cited as polymers suitable for use as the modified release component. The only therapeutic agent specifically disclosed for release as described therein was methylphenidine.

U.S. Pat. No. 6,224,910 (Ullah et al.) discloses multiparticulates in the form of enteric coated beadlets. The preferred enteric coating was Eudragit L-30-D55. The beadlets were produced using a binder in the core of each beadlet, such as sodium carboxymethyl cellulose, HPMC, or PVP. The patent notes that when capsules of such beadlets are delivered orally to a subject and the capsule shell dissolves in gastric juices, the beadlets can become tacky on moistening, in which case the beadlets do not act as a multiparticulate system. A method of overcoming this problem was disclosed, wherein a hydrophobic overcoat in the form of a dry powder, such as talc, was dusted on the surfaces of the beadlets before encapsulation.

The examples from the literature cited above illustrate a wide variety of different forms of multiparticulates that have been developed for use in oral formulations. Unfortunately, not all of the formulations are suitable for use with all therapeutic agents. For example, some heat sensitive drugs are not suitable for use in melt-extruded multiparticulates containing such drugs. Osmotic systems, such as that described by Am Ende et al. '933, are cost-prohibitive to produce for use with many therapeutic agents, due to the high cost of membranes and membrane application operations. Uncoated matrix systems do not allow for as much control of release rates as coated systems, and coated systems have the potential of sticking together when exposed to moisture. Ullah et al. '910 describes one solution to the tackiness problem, a solution suitable for use in capsule formulations, a solution wherein multiparticulates are overcoated with a dry powder such as talc. However, the addition of such a dry powder is likely to inhibit the formation of coherent compressed tablets of multiparticulates.

Some therapeutic agents, such as clindamycin, are particularly difficult to produce in the form of compressed tablets of multiparticulates due to the fact that they tend to behave like a monolithic tablet when multiparticulates thereof are directly compressed into tablets, failing to disintegrate within less than 30 minutes, as is preferred for multiparticulate tablets. Furthermore, the recommended dosage of some agents, such as clindamycin, is relatively large for an average adult human. In an unpublished study, oral compressed tablets produced by directly compressing multiparticulate clindamycin were found to demonstrate a sustained release rate. However, the tablets behaved like monolithic tablets, failing to disintegrate in a 24 hour dissolution test. As part of the same study, it was found that disintegration time could be reduced by the addition of extra-multiparticulate material to the formulation; but, a significant increase in tablet size was needed in order to incorporate the extra-multiparticulate material. The resulting tablet size was so large that oral administration to an average adult human would be impractical.

What is needed is an oral extended release multiparticulate formulation that minimizes particulate agglomeration while promoting cohesiveness of compressed formulations of the multiparticulates. What is also needed for active agents, such as clindamycin, that require relatively high drug loading is such a multiparticulate compressed formulation that minimizes the amount of extra-multiparticulate material in the formulation.

### BRIEF SUMMARY OF THE INVENTION

The present invention relates to an extended release multiparticulate composition, comprising a plurality of particulates, each comprising: a core comprising a hydrophilic therapeutic agent and a binder, a release rate controlling polymer coating the core, and a binder-dispersing agent overcoating the polymer coating.

In one embodiment, the present invention relates to a compressed tablet of the multiparticulate composition of the invention.

In another embodiment, the present invention relates to oral administration of a dose of clindamycin incorporated into the core of the multiparticulates of the compressed tablets of the invention to treat or prevent a bacterial infection.

### BRIEF DESCRIPTION OF THE DRAWING

FIG. 1 is a drug release profile of three compressed tablets produced from multiparticulate clindamycin crystalline free base particles coated with different amounts of Eudragit RS30D and overcoated with povidone, as described in Example 2.

### DETAILED DESCRIPTION OF THE INVENTION

The term “multiparticulate” means a plurality of discrete or aggregated particles, pellets, beads, granules, or mixture thereof, irrespective of their size, shape or morphology. Each individual particle, pellet, bead, or granule making up a multiparticulate is referred to herein, as a “particulate.”

The term “therapeutic agent” as used herein refers to a pharmaceutically active agent, such as a drug.

The term “binder-dispersing agent” refers to a substance of matter that acts as both a binder and as a dispersing agent, particularly, in a compressed tablet formulation. Examples of binder-dispersing agents include povidone and cross-povidone.

The term “superdisintegrant” as used herein refers to a substance that facilitates rapid disintegration at low use levels in tablets, capsules, granules, and other disintegrating dosage forms.

The term “oral administration,” as used herein, refers a form of delivery of a dosage form of a therapeutic agent to a subject, wherein the dosage form is placed in the mouth of the subject and swallowed.

The term “orally deliverable” herein means suitable for oral administration.

The term “dose unit” herein means a portion of a pharmaceutical composition that contains an amount of a therapeutic agent suitable for a single oral administration to provide a therapeutic effect. Typically, one dose unit, or a small plurality (up to about 4) of dose units, administered as a single oral administration, provides a sufficient amount of the agent to result in the desired effect.

The term “enteric coating”, as used herein, refers to a tablet coating that is resistant to gastric juice, and which dissolves after a dosage form with the enteric coating passes out of the stomach, after oral administration to a subject.

The term “excipient”, as used herein, means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, lubricants, glidants, substances added to mask or counteract a disagreeable taste or odor, flavors, dyes, fragrances, and substances added to improve appearance of the composition.

The core of each particulate of the extended release multiparticulate compositions of the present invention comprises a hydrophilic therapeutic agent and a binder. Examples of therapeutic agents suitable for inclusion as at least one therapeutic agent in the core of each particulate of the present multiparticulate compositions include, but are not limited to antihistamines, antibiotics, antituberculosis agents, cholinergic agents, antimuscarinics, sympathomimetics, sympatholytic agents, autonomic drugs, iron preparations, haemostatics, cardiac drugs, antihypertensive agents, vasodilators, non-steroidal antiinflammatory agents, opiate agonists, anticonvulsants, tranquilizers, stimulants, barbiturates, sedatives, expectorants, antiemetics, gastrointestinal drugs, heavy metal antagonists, antithyroid agents, genitourinary smooth muscle relaxants and vitamins. Examples of specific therapeutic agents suitable for use in the compositions and tablets of the present invention include reboxetine, clindamycin, (-)-S-3-(3-methylsulfonylphenyl)-N-n-propylpiperidine, sumanirole, pramipexole, atenolol, propoxyphene, metformin, metoprolol, amitriptyline, ranitidine, fexofenadine, quinapril, sildenafil, tramadol, verapamil, gabapentin, potassium chloride, alendronate, bupropion, levofloxacin, doxycycline, venlafaxine, allopurinol, isosorbide mononitrate, fosonipril, propanolol, promethazine, captopril, fluvastatin, cimetidine, sumatriptan, nortriptyline, naproxen, calacyclovir, doxepin, amoxicillin, azithromycin, diltiazem, cefprozil, acyclovir, ciprofloxacin, losartan, and pharmaceutically acceptable salts of any of said active agent. It is preferred that the active agent is selected from the group consisting of reboxetine, clindamycin, (-)-S-3-(3-methylsulfonylphenyl)-N-n-propylpiperidine hydrochloride sumanirole, pramipexole, and pharmaceutically acceptable salts of any of said active agent. The active agent is most preferably a form of clindamycin.

When a salt form of any given therapeutic agent is too soluble to provide desired extended release characteristics using a dosage form of the present invention, it may be preferred to use a less soluble form, such as a crystalline form, of the same therapeutic agent in the dosage form. When the therapeutic agent is clindamycin, the clindamycin can be present as a salt of clindamycin, such as clindamycin hydrochloride or clindamycin phosphate, or as a pharmaceutically active analog of clindamycin, such as analogs disclosed in U.S. Pat. No's 3,496,163; 4,568,741; and 3,583,972, incorporated herein by reference. When the antibiotic is clindamycin, the clindamycin is most preferably present as crystalline clindamycin free base.

Crystalline clindamycin free base is disclosed in U.S. patent application No. 10/228,356, incorporated herein by reference. Crystalline clindamycin free base can be produced by either of the two alternative processes, illustrated in the above-referenced patent application. One illustrative process of preparing crystalline clindamycin free base involves forming the amorphous free base as a precipitate in aqueous medium followed by agitation to crystallize the free base from the precipitate. An illustrative example of the method involves first dissolving a salt of clindamycin, e.g., clindamycin hydrochloride in a solvent, preferably a polar solvent such as, for example, water. This if followed by adding an alkali material, i.e. a base, in an aqueous vehicle such as for example, a NaOH solution, such as, for example, preferably from about 0.01 to about 10 N NaOH solution, more preferably from about 0.1 to about 1 N NaOH, and more preferably about 0.5 N NaOH. This results in precipitation of the amorphous free base. The amorphous free base is then crystallized by agitation of the precipitate by, for example, by sonicating or manually shaking the precipitate, or by both sonicating and manually shaking the precipitate suspended in the aqueous medium. The crystallized free base is then preferably harvested by centrifugation, followed by removal of the liquid portion. The crystallized free base is preferably washed in at least one washing step involving adding a wash solution, sonicating, shaking, centrifuging and removing the wash solution from the crystalline material. The wash solution is preferably aqueous, more preferably water.

In an alternate method, crystalline clindamycin free base can be produced by a slow addition of a clindamycin salt, such as clindamycin hydrochloride, dissolved in a polar solvent such as water to an aqueous alkaline solution containing a water-soluble organic substance, preferably an alcohol co-solvent. The aqueous solution containing an alkali with an alcohol co-solvent is prepared by adding the alkali, i.e. base, in an aqueous vehicle such as, for example, a NaOH solution. The NaOH solution can be, for example, preferably from about 0.01 to about 10 N NaOH solution, more preferably from about 0.1 to about 1 N NaOH, and more preferably about 0.5 N NaOH. The alcohol co-solvent is present, preferably in an amount of from about 2% to about 20%, more preferably from about 5% to about 10%. Any of a number of alcohols that are readily miscible with water can be used, preferably, methanol, ethanol, n-propanol, t-butanol and the like. Typically alcohols of higher molecular weight are less soluble in water and less preferred. Diols such as 1,2, ethanediol (ethylene glycol), 1,2 propanediol (propylene glycol) and 1,2 butanediol and triols such as 1,2,3 propantriol (glycerol) and the like can also be used as co-solvent. It is also possible to use an aqueous solution of a water-soluble organic substance such as, for example, sodium acetate.

An aqueous solution of a clindamycin salt, such as, for example clindamycin hydrochloride is prepared and slowly added to the alkali solution with alcohol co-solvent, preferably over a period of from about 15 minutes to about 4 hours, more preferably from about 30 minutes to about 2 hours and most preferably from about 45 minutes to 75 minutes. Crystallization is allowed to proceed for 1 to 24 hours and the crystalline free base material is isolated by filtration, centrifugation and decanting or the like. In a preferred variation of this method, the clindamycin hydrochloride solution is added in a multi-phase infusion schedule such as, for example, a first phase of slow infusion over about one hour, followed by a faster infusion phase over about 30 min and concluding with slow infusion phase over about one hour.

The material obtained by either of the methods above is isolated and dried, for example, under a stream of humidified nitrogen. The dry material can be further processed such as by grinding to produce a dry powder.

In addition to the hydrophilic drug, the core of each particulate further comprises a core binder. Examples of suitable core binders include, either individually or in combination: acacia; tragacanth; sucrose; gelatin; glucose; starches such as, but not limited to, pregelatinized starches (e.g., National™ 1511 and National™ 1500); celluloses such as, but not limited to, methylcellulose, microcrystalline cellulose, and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; hydroxypropylmethylcellulose (hereinafter, “HPMC”); hydroxypropylcellulose (e.g., Klucel™); and ethylcellulose (e.g., Ethocel™). The core binder is preferably ethylcellulose, microcrystalline cellulose, or HPMC, more preferably, ethylcellulose.

In another embodiment of the invention, each particulate core further comprises a core lubricant. Suitable core lubricants include, either individually or in combination, glyceryl behenate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. The core lubricant is preferably magnesium stearate, calcium stearate, stearic acid, and a hydrogenated vegetable oil, more preferably magnesium stearate.

In another embodiment, the particulate core further comprises an additional excipient, a buffer, a surfactant, an additional binder, or an additional lubricant. Any such additional excipients are preferably limited in amount, in order to not unduly limit the percent by weight of the core occupied by the therapeutic agent, particularly in cases where large quantities of the therapeutic agent are required in order to have a therapeutic effect on a subject to whom the composition is orally administered.

The components of the core are suitably formed into particulates by any one of a number of different means, including wet granulation, dry granulation, roller compaction, extrusion, spheronization, fluid bed granulation, co-precipitation, microencapsulation, spray-drying, melt-extrusion, and pelletization. Roller compaction is a particularly preferred means of forming the components of the core into particulates. The particulates are preferably formed by compaction, more preferably by roller compaction. Roller compaction has an advantage of enabling one to press materials into particulates that are substantially uniform in size. Greater uniformity in size makes it more likely that a composition of multiparticulates will release a therapeutic agent from the core at a more uniform rate.

Regardless of what means is used to produce the particulate core particles, the particles are preferably screened to ensure greater particle size uniformity. The particular particle size range selected depends upon desired flow characteristic of the particles, content uniformity, and surface area desired. The individual particle size of particulates in the composition of the present invention is preferably between about 10 μm to about 2 mm, more preferably between about 50 μm to about 2 mm, most preferably between about 100 μm to about 1.5 mm. The average particle size of the plurality of particulates in the composition of the present invention is preferably about 250 μm to about 1.2 mm.

The core of each particulate of the present invention can suitably be coated by any one of a number of known means, preferably through an aqueous dispersion of the polymer coating or after dissolution in an appropriate solvent. The method used to coat any given set of particulates depends upon the chemical and physical characteristics of the components of the particulate cores, including conditions under which the therapeutic agent is likely to loose therapeutic efficacy.

Release rate controlling polymer coatings suitable for use in coating the multiparticulate components of the compositions of the present invention include, but are not limited to, vinyl acetate, vinyl chloride, vinyl carbonate, methacrylic acid, polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate. Ethyl cellulose, cellulose acetate phthalate methacrylic acid, and polymethacrylic acid copolymer are preferred, with methacrylic acid, and polymethacrylic acid copolymers being particularly preferred. Even more particularly preferred are copolymers of acrylate and methacrylate with quaternary ammonium groups, such as the Eudragit® RS and RL (Rohm & Haas) series of copolymers.

Eudragit® RS30D is the most particularly preferred release rate controlling polymer coating for various reasons. First, it provides an extended release rate of hydrophilic therapeutic agents, such as clindamycin, from a core coated therewith. Second, it can be applied at room temperature, making it easy to manufacture and coat cores therewith. The polymer is also sufficiently flexible to allow for compaction without cracking.

The selection of the type and characteristics of the release rate controlling polymer coating can affect the rate of release of the therapeutic agent from each particulate. A thicker polymer coating layer will extend the release of the therapeutic agent therefrom more effectively than a thinner polymer coating layer. A pore-forming agent, such as hydroxypropyl methyl cellulose in the release rate controlling polymer coating can speed up the therapeutic agent release rate. Enteric coatings delay release of the therapeutic agent until after multiparticulates coated therewith pass from a lower pH environment, such as is found in the upper gastrointestinal tract of a mammal, into closer to a neutral pH environment, such as is found in the lower gastrointestinal tract.

In one embodiment of the present invention, all of the multiparticulates are coated with the same type and with substantially the same amount of the release rate controlling polymer coating.

In another embodiment, the multiparticulate composition of the present invention suitably comprises a plurality of particulates at least two of which particulates have a release rate controlling polymer coating that differs in type or physical characteristics from the other. The at least two particulates with different coatings preferably have coatings of two different thicknesses.

The binder-dispersing agent overcoating the polymer coating of each particulate of the multiparticulate composition of the present invention performs two functions. It acts as a binding agent, enhancing cohesion when the multiparticulates are compacted with extra-multiparticulate material, such as is done in the formation of the tablets of the present invention. The binder-dispersing agent also acts as a dispersing agent, ensuring that the multiparticulates do not agglomerate when the multiparticulate composition comes into contact with an aqueous solution, such as tablets of the composition do in the gastrointestinal tract of a subject after oral administration thereto. The most preferred binder-dispersing agents for use in the compositions of the present invention provide a balance between these two functions, allowing one to produce a coherent tablet of the multiparticulate composition that releases the therapeutic agent at a rate controlled by the multiparticulate particles acting independently of one another rather than as one or more agglomerated cohesive mass. Suitable binder-dispersing agents for use in overcoating the polymer coating of the multiparticulate cores include polyvinylpyrrolidone (“povidone”) and derivatives thereof, preferably povidone or cross-povidone.

In another embodiment, the extended release multiparticulate composition of the present invention further comprises extra-multiparticulate material compressably commingled with the plurality of multiparticulates. The most preferred form of this embodiment of the invention is a compressed tablet.

In the embodiment of the composition described immediately above, the at least one extra-multiparticulate material preferably comprises an extra-multiparticulate binder. The extra-particulate binder preferably imparts sufficient cohesion to the multiparticulates and extra-multiparticulate material being tableted to allow for normal processing operations such as sizing, lubrication, compression and packaging, but still allow the tablet to disintegrate and the composition to be absorbed upon ingestion. Suitable extra-multiparticulate binders include, either individually or in combination, acacia; tragacanth; sucrose; gelatin; glucose; starches such as, but not limited to, pregelatinized starches (e.g., National™1511 and National™ 1500); celluloses such as, but not limited to, methylcellulose, microcrystalline cellulose, and carmellose sodium (e.g., Tylose™ ); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). An extra-multiparticulate binder, if present, constitute in total about 0.5% to about 25%, preferably about 0.75% to about 15%, and more preferably about 1% to about 10%, of the total weight of the composition.

When the therapeutic agent is clindamycin, microcrystalline cellulose is a particularly preferred extra-multiparticulate binder, because of its known chemical compatibility with that particular drug. The use of extra-multiparticulate microcrystalline cellulose can also be used to improve hardness and/or disintegration time. Microcrystalline cellulose included in dry granulation similarly improves hardness of a tablet core.

In addition to a binder, the extra-multiparticulate material of the multiparticulate composition of the present invention preferably further comprises one or more pharmaceutically acceptable extra-multiparticulate lubricant. Suitable extra-multiparticulate lubricants include, either individually or in combination, glyceryl behenate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4 and Carbowax™ 6000); sodium oleate; sodium lauryl sulfate; and magnesium lauryl sulfate. Such extra-multiparticulate lubricants, if present, constitute in total about 0.1% to about 10%, preferably about 0.2% to about 8%, and more preferably about 0.25% to about 5%, of the total weight of the composition.

Magnesium stearate is a preferred extra-multiparticulate lubricant used, for example, to reduce friction between the equipment and the mixture of multiparticulates and extra-multiparticulate material during compression of the compositions of the present invention to form compressed tablets.

In another embodiment, the extended release multiparticulate composition of the present invention optionally further comprises a buffer. When a buffer is present, it is preferably a buffer designed to maintain the pH at a pH range wherein the therapeutic agent is stable.

In yet another embodiment, the composition of the present invention optionally further comprises one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 10% to about 80%, of the total weight of the composition. The diluent or diluents selected preferably exhibit suitable flow properties and, where tablets are desired, compressibility.

Other excipients such as colorants, flavors, sweeteners, and disintegrants are known in the pharmaceutical art and can be used in compositions of the present invention. Suitable disintegrants include: either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, cross-povidone, and gums such as agar, guar, locust bean, karaya, pectin and tragacanth gums. When a disintegrant is present, it is preferably a superdisintegrant, more preferably, croscarmellose sodium.

The multiparticulates of the extended release tablet of the present invention preferably contain a therapeutic amount of the therapeutic agent. How much of any given therapeutic agent constitutes a therapeutic amount for a given subject is dependent inter alia on the body weight of the subject. Where the therapeutic agent is clindamycin, and the subject is a child or a small animal (e.g., a dog), for example, an amount of clindamycin relatively low in the preferred range of about 24 mg/kg/day to about 80 mg/kg/day. An especially preferred amount of clindamycin crystalline free base per dosage form is typically about 24 mg/kg/day to about 64 mg/kg/day, which is likely to provide blood serum concentrations consistent with therapeutic effectiveness. Where the subject is an adult human or a large animal (e.g., a horse), achievement of such blood serum concentrations of clindamycin or of another therapeutic agent are likely to require dose units containing a relatively greater amount of the therapeutic agent. For an adult human, a therapeutically effective amount of crystalline clindamycin free base per dosage form in a composition of the present invention is suitably about 500 mg to about 2 mg, more preferably about 600 mg to about 1800 mg. An especially preferred amount of crystalline clindamycin free base per dosage form for an adult human is about 600 mg to about 1200 mg.

The amount of therapeutic agent in a given dosage form can be selected to accommodate the desired frequency of administration used to achieve a specified daily dosage. The amount of the unit dosage form of the composition that is administered and the dosage regimen for treating the condition or disorder will depend on a variety of factors, including the age, weight, sex and medical condition of the subject, the severity of the condition or disorder, the route and frequency of administration, and the particular therapeutic agent selected, and thus may vary widely. One or more dosage forms can be administered up to about 6 times a day. However, the dosage forms of the present invention release at an extended rate, making it possible to provide the desired therapeutic efficacy by administration once-a-day or twice-a-day.

In a particularly preferred embodiment, the invention relates to a compressed tablet, comprising a plurality of particulates, each particulate comprising: (a) a core comprising clindamycin crystalline free base and ethylcellulose; (b) a release rate controlling polymer coating the core; (c) povidone or a derivative thereof overcoating the release rate controlling polymer coating; and extra-multiparticulate material comprising a cellulose derivative, magnesium stearate, and a superdisintegrant. The cellulose derivative is preferably selected from the group consisting of ethylcellulose, microcrystalline cellulose, and HPMC.

In an alternative embodiment, the compressed tablet of the present invention is coated with a tablet polymer coating. Suitable non-functional polymer coatings include HPMC (e.g., Opadry® coating), polyvinylacetate, PVP, carageenen based, or other non-functional film coats. Suitable hydrophobic polymers for use as the tablet polymer coating include hydroxypropylcellulose, hydroxyethylcellulose, methylhydroxyethylcellulose, methylcellulose, ethylcellulose (e.g., Surelease™ of Colorcon), cellulose acetate, sodium carboxymethylcellulose, polymers and copolymers of acrylic acid and methacrylic acid and esters thereof (e.g., Eudragit™ RL, Eudragit™ RS, Eudragit™ L100, Eudragit™ S100, Eudragit™ NE), and Acryl-ese® (Colorcon), polyvinylpyrrolidone, and polyethylene glycols. The polymers can be combined with water-soluble polymers, such as HPMC or polyethylene glycol to form pores or channels in the coating to modify the release rate. In an alternative embodiment, the tablet polymer coating is comprised of an enteric coating (e.g., a Sureteric® coating) or a pH independent coating.

Another embodiment of the present invention is directed to a method of treating or preventing a condition by oral administration of at least one compressed tablet of the present invention to a subject. The subject is preferably a mammal, more preferably a mammal selected from the group consisting of a cat, a dog, and a human being. Even more preferably, the subject is a human being. The exact therapeutic agent delivered through the tablet to a given subject depends upon the condition to be treated or prevented by the dosage form. For example, when the subject is infected with or in danger of being infected with one or more strains of bacteria, at therapeutic agent is an antibiotic. The tablet administered could also suitably include more than one drug, such as an antibiotic and an anti-pain medication.

When at least one active agent in the composition and method of using tablets of the compositions of the present invention is clindamycin, the invention is useful in treatment and prevention of a wide range of bacterial infections. Such compositions and methods can be used for the treatment of serious infections caused by susceptible gram-positive bacteria, such as streptococci, pneumococci, and staphylococci, for example, that cause serious respiratory tract infections such as empyema, anaerobic pneumonitis and lung abscess; serious skin and soft tissue infections; septicemia; intra-abdominal infections such as peritonitis and intra-abdominal abscess (typically resulting from anaerobic organisms resident in the normal gastrointestinal tract); infections of the female pelvis and genital tract such as endometritis, nongonococcal tubo-ovarian abscess, pelvic cellulitis and postsurgical vaginal cuff infection.

The present invention is further illustrated by the following examples. These examples are intended to be illustrative of the invention and should not be used to limit or restrict its scope.

### EXAMPLES

### Example 1

Use of Various Overcoats of Compressed Tablets

Compressed tablets were produced from multiparticulates of clindamycin coated with Eudragit RS30D as a rate controlling polymer and overcoated with HPMC, polyvinyl acetate, or povidone.

HPMC was found to be a poor binder.

Polyvinyl acetate was found to be an acceptable binder, but a poor dispersant.

The povidone overcoat was found to reduce tackiness and aid in tablet compression. Tablets of multiparticulates over coated with the povidone were of an acceptable hardness, yet disintegrated readily when placed in an aqueous environment.

### Example 2

Production of Tablets with Multiparticulates Overcoated with Varying Amounts of Povidone

Tablets of multiparticulates of clindamycin crystalline free base coated with varying amounts of coating material (for 7%, 10%, and 15% weight gain) and overcoated with povidone (for a 7.5% weight gain) were produced and tested as described below:

TABLE 1

| | | | |
|---|---|---|---|
| 7% Weight | 10% Weight | 15% Weight | |
| Gain | Gain | Gain | |
| mg. (% weight) | mg. (% weight) | mg. (% weight) | Component |

Granulation Phase (Production of Core)

| | | | | | | |
|---|---|---|---|---|---|---|
| 600* | (73.63%) | 600* | (71.62%) | 600* | (68.51%) | Clindamycin crystalline free |
| | | | | | | base |
| 106.7* | (13.1%) | 106.7* | (12.7%) | 106.7* | (12.2%) | Ethylcellulose |
| 1.77 | (0.22%) | 1.77 | (0.21%) | 1.77 | (0.20%) | Magnesium Stearate |

Coating

| | | | | | | |
|---|---|---|---|---|---|---|
| 32.95 | (4.04%) | 47.07 | (5.6%) | 70.61 | (8.06%) | Eudragit RS30D |
| 9.89 | (1.21%) | 14.12 | (1.69%) | 21.18 | (2.42%) | Micronized Talc |
| 0.165 | (0.02%) | 0.235 | (0.028%) | 0.353 | (0.04%) | Simethicone |
| 6.59 | (0.81%) | 9.41 | (1.12%) | 14.12 | (1.61%) | Triethyl Citrate |

| | | | |
|---|---|---|---|
| 198.38 | 238.22 | 425.1 | Purified Water USP |

Overcoat

| | | | | | | |
|---|---|---|---|---|---|---|
| 56.85 | (6.89%) | 58.45 | (6.98%) | 61.1 | (6.98%) | PVP K-30 |

| | | | |
|---|---|---|---|
| 511.65 | 525.96 | 549.9 | Purified Water USP |
| 814.92 | 837.76 | 875.83 | Total Weight |

The components of the granulation phase of each formulation shown in Table 1 were mixed together and roller compacted to form the core of each multiparticulate, according to the following procedure:

1. All intragranular ingredients except magnesium stearate were weighed.

2. The same ingredients from step 1 were sized through a suitably sized mesh hand screen.

3. The same ingredients were then dry mixed in a suitable blender (a PK blender (Patterson Kelley), in this case) for 7 minutes.

4. The intragranular portion of magnesium stearate (screened through a 30 mesh screen) was weighed and manually blended with a portion of the mixture from step 3, above.

5. The manually blended mixture from step 4 was then combined in a blender with the remainder of the mixture from step 3, and mixed for an additional 3 minutes.

6. The intragranular mixture resulting from step 5 was then run through a roller compactor to achieve a suitable ribbon. Initial granulation was performed by an Alexanderwerk.

7. The material from the first granulation step was separated by sieving using the appropriate mesh screens. Material that meets the predetermined particle size specification was collected. A 16/40 mesh cut was collected (material that passed through a 16 mesh screen, but was retained on a 40 mesh screen). The particle size range was approximately 250 μm to 1.2 mm.

8. The overs from step 7 were milled again using a suitable mill (e.g., a Fitzmill (The Fitzpatrick Company)

9. Steps 6-8 were repeated three times, or until an acceptable yield of multiparticulate cores was obtained.

10. The multiparticulate cores were then coated with one of the three coating mixtures shown in Table 1.

11. The coated multiparticulate cores were overcoated with a suspension of PVP in water according to the formulae of Table 1.

Tablets of each of the three types of multiparticulates produced as described above were produced by mixing each set of multiparticulates with an extra-multiparticulate mix of: 200 mg microcrystalline cellulose, 40.0 mg croscarmelose sodium, and 1.6 mg of magnesium stearate, according to the following remaining steps of the procedure:

12. All extra-multiparticulate ingredients except microcrystalline cellulose were weighed. The weight was adjusted to match the yield of material obtained in step 10.

13. The extra-multiparticulate ingredients weighed in step 12 were dry mixed with the multiparticulates in a suitable blender (e.g., a PK blender) for 7 minutes.

14. The extra-multiparticulate magnesium stearate (screened through a 30 mesh screen) was weighed and manually blended with a portion of the mixture of step 12.

15. The premixed ingredients from step 14 were combined with the mixture from step 12 and mixed for an additional 3 minutes.

16. Samples of the resulting mixture from step 15 were compressed into tablets.

### Example 3

Testing of Tablets

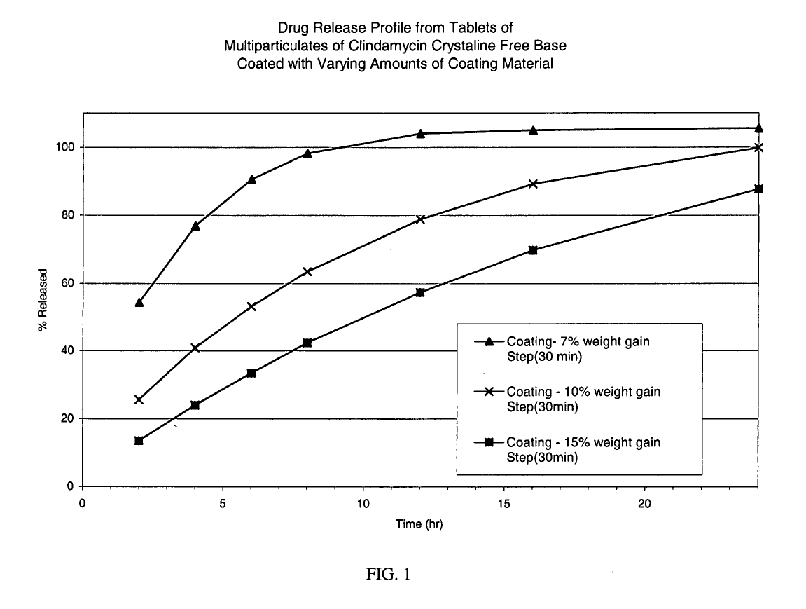

The three types of compressed tablets produced as described in Example 3 were tested for the rate of release of clindamycin from samples of each tablet in an aqueous solution of 0.05 M phosphate (pH 6.35, made from potassium phosphate salts) and 0.1 M chloride (HCI), wherein the pH of the solution was retained at pH 2 for 30 minutes and then raised to pH 6.35 for the remainder of the assay. The ionic strength of the aqueous solution was 0.1 for the first 2 hours of the assay, at which point it was raised to 0.175. A drug release profile of the assay results is shown in FIG. 1.

As one can see from FIG. 1, the tablets with multiparticulates coated with the thickest coating released the least amount of clindamycin at each time point, for the greatest extension of time of release of the three formulations tested. Specifically, the tablets produced from multiparticulates coated for a 15% weight gain showing the greatest extension of release of clindamycin over time, with those coated for a 10% weight gain showing a little less extension of time of drug release, and those coated for a 7% weight gain showing the least extension of time of drug release of the three formulations tested. 16 hours after initiation of the assay, the 90% of the clindamycin had been released from the tablets with multiparticulates coated for a 10% weight gain, while only 70% of the clindamycin had been released from those coated for a 15% weight gain, and only about 60% of the clindamycin had been released from those coated for a 7% weight gain.

We claim:

1. An extended release multiparticulate composition, comprising:
a plurality of particulates, each comprising:
a core comprising a hydrophilic theraputic agent and a core binder, a release rate controlling polymer coating covering the core and a binder-dispersing agent over-coating the polymer coating.

2. The extended release composition of claim 1, wherein the plurality of particulates have an average particle size of about 250 μm to about 1.2 mm.

3. The extended release composition of claim 1, wherein the hydrophilic therapeutic agent is selected from the group consisting of: reboxetine, clindamycin, (-)-S-3-(3-methylsulfonylphenyl)-N-n-propylpiperidine, sumanirole, pramipexole, atenolol, propoxyphene, metformin, metoprolol, amitriptyline, ranitidine, fexofenadine, quinapril, sildenafil, tramadol, verapamil, gabapentin, potassium chloride, alendronate, bupropion, levofloxacin, doxycycline, venlafaxine, allopurinol, isosorbide mononitrate, fosonipril, propanolol, promethazine, captopril, fluvastatin, cimetidine, sumatriptan, nortriptyline, naproxen, calacyclovir, doxepin, amoxicillin, azithromycin, diltiazem, cefprozil, acyclovir, ciprofloxacin, losartan, and pharmaceutically acceptable salts of any of said active agent.

4. The extended release composition of claim 1, wherein the hydrophilic therapeutic agent is a salt of clindamycin.

5. The extended release composition of claim 4, wherein the salt of clindamycin is selected from the group consisting of clindamycin hydrochloride, clindamycin phosphate, and clindamycin palmitate.

6. The extended release composition of claim 1, wherein the hydrophilic therapeutic agent is a free base of clindamycin.

7. The extended release composition of claim 6, wherein the free base of clindamycin is clindamycin crystalline free base.

8. The extended release composition of claim 1, wherein the clindamycin is about 20% to about 90% by weight of the plurality of particulates.

9. The extended release composition of claim 1, wherein the core binder comprises ethylcellulose.

10. The extended release composition of claim 1, the core further comprising a core lubricant.

11. The extended release composition of claim 10, wherein the lubricant is magnesium stearate.

12. The extended release composition of claim 1, wherein the release rate controlling polymer coating is an extended release polymer coating.

13. The extended release composition of claim 12, wherein the extended release polymer coating is a copolymer of acrylate and methacrylate with quaternary ammonium groups.

14. The extended release composition of claim 13, wherein the copolymer of acrylate and methacrylate is Eudragit® D RS30D or an equivalent thereof.

15. The extended release composition of claim 1, wherein the binder-dispersing agent over-coating the polymer coating is povidone or a derivative thereof.

16. The extended release composition of claim 15 wherein the povidone or a derivative thereof is in a form selected from the group consisting of povidone and cross-povidone.

17. The extended release composition of claim 1, further comprising extra-multiparticulate material compressably commingled with the plurality of particulates.

18. The extended release composition of claim 17, the extra-multiparticulate material comprising an extra-multiparticulate binder.

19. The extended release composition of claim 18, wherein the extra-multiparticulate binder is a cellulose derivative.

20. The extended release composition of claim 19, wherein the cellulose derivative is selected from the group consisting of ethylcellulose, hydroxypropylmethylcellulose, and microcrystalline cellulose.

21. The extended release composition of claim 17, the extra-multiparticulate material comprising an extra-multiparticulate lubricant.

22. The extended release composition of claim 21, wherein the extra-multiparticulate lubricant is magneseium stearate.

23. The extended release composition of claim 17, the extra-multiparticulate material comprising a disintegrant.

24. The extended release composition of claim 23, wherein the disintegrant is a superdisintegrant.

25. The extended release composition of claim 24, wherein the superdisintegrant is croscarmellose sodium.

26. The extended release composition of claim 17, the extra-multiparticulate material further comprising a surfactant.

27. A compressed tablet, comprising:
a plurality of particulates, each comprising:
a core comprising a hydrophilic therapeutic agent and a core binder,
a release rate controlling polymer coating covering the core, and
a binder-dispersing agent overcoating the release rate controlling polymer coating; and
extra-multiparticulate material compressibly commingled with the plurality of particulates, the extra-multiparticulate material comprising a superdisintegrant.

28. The compressed tablet of claim 27, wherein the plurality of particulates have an average particle size of about 250 μm to about 1.2 mm.

29. The compressed tablet of claim 27, wherein the hydrophilic therapeutic agent is selected from the group consisting of: reboxetine, clindamycin, (-)-S-3-(3-methylsulfonylphenyl)-N-n-propylpiperidine, sumanirole, pramipexole, atenolol, propoxyphene, metformin, metoprolol, amitriptyline, ranitidine, fexofenadine, quinapril, sildenafil, tramadol, verapamil, gabapentin, potassium chloride, alendronate, bupropion, levofloxacin, doxycycline, venlafaxine, allopurinol, isosorbide mononitrate, fosonipril, propanolol, promethazine, captopril, fluvastatin, cimetidine, sumatriptan, nortriptyline, naproxen, calacyclovir, doxepin, amoxicillin, azithromycin, diltiazem, cefprozil, acyclovir, ciprofloxacin, losartan, and pharmaceutically acceptable salts of any of said active agent

30. The compressed tablet of claim 27, wherein the hydrophilic therapeutic agent is a salt of clindamycin.

31. The compressed tablet of claim 30, wherein the salt of clindamycin is selected from the group consisting of clindamycin hydrochloride, clindamycin phosphate, and clindamycin palmitate.

32. The compressed tablet of claim 27, wherein the hydrophilic therapeutic agent is a free base of clindamycin.

33. The compressed tablet of claim 32, wherein the free base of clindamycin is clindamycin crystalline free base.

34. The compressed tablet of claim 27, wherein the clindamycin is about 20% to about 90% by weight of the plurality of particulates.

35. The compressed tablet of claim 27, wherein the core binder comprises ethylcellulose.

36. The compressed tablet of claim 27, the core further comprising a core lubricant.

37. The compressed tablet of claim 36, wherein the core lubricant is magnesium stearate.

38. The compressed tablet of claim 27, wherein the release rate controlling polymer coating is an extended release polymer coating.

39. The compressed tablet of claim 38, wherein the extended release polymer coating is a copolymer of acrylate and methacrylate with quaternary ammonium groups.

40. The compressed tablet of claim 39, wherein the copolymer of acrylate and methacrylate is Eudragit® RS30D or an equivalent thereof.

41. The compressed tablet of claim 27, wherein the binder-dispersing agent over-coating the polymer coating is povidone or a derivative thereof.

42. The compressed tablet of claim 41 wherein the povidone or a derivative thereof is in a form selected from the group consisting of povidone and cross-povidone.

43. The compressed tablet of claim 27, the extra-multiparticulate material further comprising an extra-multiparticulate binder.

44. The compressed tablet of claim 43, wherein the extra-multiparticulate binder is a cellulose derivative.

45. The compressed tablet of claim 44, wherein the cellulose derivative is selected from the group consisting of ethylcellulose, hydroxypropylmethylcellulose, and microcrystalline cellulose.

46. The compressed tablet of claim 27, the extra-multiparticulate material further comprising an extra-multiparticulate lubricant.

47. The compressed tablet of claim 46, wherein the extra-multiparticulate lubricant is magneseium stearate.

48. The compressed tablet of claim 27, the extra-multiparticulate material further comprising a surfactant.

49. A method of treating or preventing a gram-positive bacterial infection in a subject according to steps comprising:
orally administering to the subject an extended release multiparticulate tablet, comprising:
a plurality of particulates, each comprising:
a core comprising clindamycin and a core binder,
a release rate controlling polymer coating covering the core, and
a binder-dispersing agent overcoating the release rate controlling polymer coating; and
extra-multiparticulate material compressably commingled with the plurality of particulates, the extra-multiparticulate comprising a superdisintegrant.

50. The method of claim 49, wherein the hydrophilic therapeutic agent is a salt of clindamycin.

51. The method of claim 50, wherein the salt of clindamycin is selected from the group consisting of clindamycin hydrochloride, clindamycin phosphate, and clindamycin palmitate.

52. The method of claim 49, wherein the hydrophilic therapeutic agent a free base of clindamycin.

53. The method of claim 52, wherein the free base if clindamycin is clindamycin crystalline free base.

54. The method of claim 49, wherein the superdisintegrant is croscarmellose sodium.

55. The method of claim 49, wherein the subject is a mammal.

56. The method of claim 49, wherein the mammal is a human being.

57. The method of claim 49, wherein the gram-positive bacteria infection treated or prevented by the method of treatment is due to a bacteria species of a genus selected from the group consisting of: streptococci, pneumococci, and staphylococci.
